(19) United States
(12) Patent Application Publication
(10) Pub. No.: US 2006/0039880 A1
(43) Pub. Date: Feb. 23, 2006

(54) Make-up composition enabling the make-up of keratin fibers, particularly eyelashes, by droplet deposition

(76) Inventors: Rosemany Gouaisbault, Orleans (FR); Melanie Faure-Tromeur, Cercottes (FR); Annie Kuentz-Mura, Faye Aux Loges (FR); Jean-Francois Tranchant, Marigny Les Usages (FR)

Correspondence Address: NATH & ASSOCIATES, 112 South West Street, Alexandria, VA, 22314, US

(21) Appl. No.: 10/527,745

(22) PCT Filed: Sep. 18, 2003
(86) PCT No.: PCT/FR03/02747

§ 371 Date: Mar. 14, 2005

(30) Foreign Application Priority Data
Sep. 19, 2002 (FR) 02/11588

(51) Int. Cl. A61K 8/89

(52) U.S. Cl. 424/70.11

(57) ABSTRACT

The invention relates to a make-up composition. This composition is characterised in that it comprises a polymer or mixture of polymers selected from the family of hydroxylated polydimethylsiloxanes or non-hydroxylated polydimethylsiloxanes, and of their mixtures, and which has a viscoelasticity and a viscosity which are well determined, in order to enable the formation of drops on keratin fibres, said polymer or mixture of polymers being dispersed in a volatile solvent, said composition not containing any product having a viscoelasticity-modifying effect, which can prevent the formation of said drops at the concentration used. The invention enables drops, which are preferably transparent, to be formed at the tips of keratin fibres, particularly eyelashes or the hair.

The present invention relates to the field of make-up of keratin fibres, particularly of eyelashes.

More specifically, the invention relates to a novel method of make-up of keratin fibres, according to which a composition is applied on these fibres which enables forming drops on these fibres, which drops are preferably transparent, shiny, which dry rapidly without sticking together, and which persist with time, and this leads to a purely aesthetic effect being obtained due to the formation of these transparent drops.

The invention also relates to novel compositions which enable this formation of drops, preferably transparent drops, to be obtained upon their application on eyelashes or the hair.

Patent Application EP 0 953 332 describes a composition for the make-up of keratin fibres, notably of eyelashes and of the hair, which contains an aqueous polymer dispersion and its use for the make-up of keratin fibres. This composition, on the condition that it be applied under particular conditions, enables a discontinuous deposit to be made on said keratin fibres which are roughly longitudinal in the form of successive droplets. The polymers used for making these compositions have viscosities which are typically of the order of those of aqueous media, and this explains that it is necessary, in order for a sufficient viscosity to be obtained, to add viscosifying agents into the compositions in order for the formation of drops on the keratin fibre to be obtained.

The inventors of the present invention have now discovered that it was possible to make compositions which enable drops to be deposited on keratin fibres, particularly on eyelashes and the hair, without having need to recourse to viscosifying agents in the composition.

More specifically, this invention results from pieces of research by the inventors on polymers having particular rheological properties, and, in particular, on silicone polymers.

It is well-known that the problems linked to the wettability of the substrates are particularly complex, both from a theoretical point of view as well as from a practical point of view, and the study of the properties of the liquid/solid interface is the subject of numerous pieces of fundamental and applied research, the industrial and economic interests in which are numerous.

No study has hitherto sought to elucidate the problem consisting in forming drops or droplets having the properties mentioned above on keratin fibres.

This aptitude to make such a formation of drops depends not only upon the nature of the substrate but also upon the nature of the composition to be deposited, and upon the amount deposited.

The inventors of the present invention have established that it was possible to attain the effect sought after by making use of a composition which contains, as essential constituents, a volatile solvent and a polymer having particular rheological properties (viscoelasticity, viscosity).

Thus, according to a first aspect, the invention relates to a novel method of make-up which makes use of such compositions to form drops on keratin fibres, particularly on eyelashes and the hair.

Further, the invention relates to novel compositions which can be used in this application, particularly compositions which are liquid at ambient temperature.

More specifically, the invention relates to a method of make-up of keratin fibres, particularly of eyelashes or the hair, intended to form drops on these fibres, characterised in that it comprises applying, onto said fibres, a composition containing a polymer or mixture of polymers selected from the family of hydroxylated polydimethylsiloxanes or non-hydroxylated polydimethylsiloxanes, and which has:
a viscoelasticity characterised by a conservation modulus G′ and a loss modulus G″, which are such that G′ be less than G″ for frequencies of lower than 0.3 Hz and greater than G″ for frequencies of higher than 3 Hz, the two curves representing G′ and G″ as a function of the frequency having a point of intersection in the interval between 0.3 and 3 Hz, preferably between 0.5 and 1.5 Hz, more preferably neighbouring 1 Hz, and
a dynamic viscosity of between 4,000 and 10,000 Pa·s at 25° C.;
   dispersed in a volatile solvent,
   said composition not containing any product having a viscoelasticity-modifying effect, which can prevent the formation of said drops at the concentration used.

According to another embodiment of the invention, the invention relates also to a method of make-up of keratin fibres, particularly of eyelashes or of the hair, intended to form drops on these fibres, characterised in that it comprises applying, onto said fibres, a composition which essentially consists of, or which consists of, a polymer or mixture of polymers selected from the family of hydroxylated polydimethylsiloxanes or non-hydroxylated polydimethylsiloxanes, and of their mixtures, and which has:
a viscoelasticity characterised by a conservation modulus G′ and a loss modulus G″, which are such that G′ be less than G″ for frequencies of lower than 0.3 Hz and greater than G″ for frequencies of higher than 3 Hz, the two curves representing G′ and G″ as a function of the frequency having a point of intersection in the interval between 0.3 and 3 Hz, preferably between 0.5 and 1.5 Hz, more preferably neighbouring 1 Hz, and
a dynamic viscosity of between 4,000 and 10,000 Pa·s at 25° C.; dispersed in a volatile solvent.

Thus, the invention essentially results from the correlation which is established by the inventors between the rheological properties of the polymers and the possibility of obtaining drops on the keratin fibres by applying, on these fibres, a solution of these polymers in a volatile solvent.

All the measurements of dynamic viscoelasticity and viscosity given in the present document are made at a temperature of the order of 25° C., with a Rheometric Dynamic Analyser RDAII apparatus which is marketed by the company Rheometrics, USA, and in following the procedure and the use notice from the constructor. As is well-known, the measurements of viscosity are measurements which are non-destructuring which are made on a Newtonian plateau whereon it is well known that the value of the frequency or that of the constraint applied to the system can be fixed alternatively.

The rheometer used for making the measurements within the context of the present invention has parallel plate geometry. The measurements which are necessary are then made at constant deformations or constant frequencies.

It is recalled that the polymers retained are those the linear viscoelasticity curve of which has a plateau which corresponds to a dynamic viscosity of between 4,000 and 10,000 Pa·s.

It will be noted that the characteristics of viscoelasticity of the polymers are:
their conservation modulus G′ which characterises their elasticity, and
their loss modulus G′ which characterises their viscosity.

The polymers retained are those for which the curves representing the moduli G′ and G″ as a function of the frequency have a point of intersection in the interval between 0.3 Hz and 3 Hz, preferably between 0.5 and 1.5 Hz, more preferably neighbouring 1 Hz.

It does in fact appear, for frequencies lower than the interval retained, that these polymers are in a state which is too liquid and, as a consequence, flow, since the polymer chains have a tendency to disentangle.

On the other hand, above the interval and these polymers go through a state which is too rubbery.

It will also be noted that the viscosity curves translate measurements made in dynamic mode and represent the deformation on the abscissa and the dynamic viscosity along the ordinate.

All the polymers which have the above rheological characteristics set forth above can be used for the preparation of the compositions which are useful according to the invention.

According to an advantageous variant however, silicone type polymers will be used and these silicones will preferably be selected from the family of hydroxylated polydimethylsiloxanes or non-hydroxylated polydimethylsiloxanes, and of their mixtures.

Dimethiconols, i.e. silicone type polymers having hydroxylated groups, and, particularly, dimethiconols having a viscosity of the order of 6,400 Pa·s at 25° C., will be selected as preferred polymers according to the invention.

The polymer dimethiconol, which is marketed by Dow Corning, notably under the name of SGM-36®, having a viscosity of 6,400 Pa·s at 25° C., will preferably be selected as an example.

The volatile solvents used for the preparation of the compositions which are used according to the invention can advantageously be any volatile solvent which is usually used in cosmetics insofar as it disperses the polymer selected and is compatible with the formation of drops.

A product which evaporates on the skin at ambient temperature will preferably be selected as a volatile solvent.

This volatile solvent will preferably have a saturating vapour pressure at atmospheric pressure and ambient temperature of less than 3 Pa.

A volatile solvent of linear or cyclic silicone type, such as linear dimethicones having 2 to 9 silicon atoms, cyclomethicones having 3 to 8 silicon atoms, will preferably be selected.

The preferred volatile solvent is hexamethyldisiloxane which enables transparent, shiny drops to be obtained which dry rapidly without sticking together and which persist with time.

The volatile solvent marketed by Dow Corning under the name of Dow Corning 200 0.65 cts will preferably be selected as an example.

Thus, according to an embodiment of the invention, anhydrous compositions can be prepared.

The proportions of polymer or of mixture of polymers and of volatile solvent which are contained in the compositions used according to the invention can vary over wide ranges. The preferred concentration of polymer(s) is however 5 to 30%, and preferably 10 to 25% by weight with respect to the weight of the make-up composition. Preferably, the concentration of polymer(s) is 15 to 25% by weight with respect to the weight of the make-up composition.

As set forth above, one of the characteristics sought after for the compositions which are used according to the invention is to lead, upon their application on keratin fibres, to drops or droplets which do not stick together.

This non-sticky character can be obtained by a selection of the polymer. It can be advantageous to introduce a product into the composition which is intended to reduce the sticky character of the drops.

In order to fulfil this function, a product which is usually used for improving the <<without transfer>> character of cosmetic compositions can advantageously be selected.

An example of a product which is particularly advantageous to this end is constituted of a mixture of a dimethicone polymer which is cross-linked by vinyldimethicone, in a solvent which is constituted by cyclomethicone D5, having a 5-membered silicon ring, said product being preferably at a concentration of between 5 and 15% by weight with respect to the weight of the composition.

An example of such a product is the product which is marketed by Shin-Etsu Silicones Europe B.V under the commercial name of KSG-15® which is a mixture of cross-linked polymers which are contained in proportions of 2 to 10% of cross-linked dimethicone polymer and 90 to 98% of cyclomethicone D5.

Thus, the cosmetic compositions or make-up compositions which are obtained have a remarkable aesthetic effect in the light due to the deposit on the eyelashes of drops, and which drops preferably have a transparent character.

According to another variant, the compositions of the invention can also be applied on the hair with the same aim of fixing drops.

According to yet another variant of the invention, it will be possible to introduce into the composition a cosmetically-acceptable additive which is non-viscoelasticity-modifying at the concentration used, such as a colouring agent, a perfuming agent, a preserving agent, an anti-oxidising agent, a UV-filter, etc.

According to a variant of the invention, the formation of drops which are transparent and coloured might be sought after.

It will be possible for this colouration to be obtained by introducing, into the compositions used, a soluble organic colouring agent and/or powdery inorganic fillers, advantageously in multilayers or composites, having visual effects such as a pearlescent effect.

This colouration of the drops will enable particular light effects to be obtained, which contribute to the aesthetic character of the drops.

According to a second aspect, the present invention relates also to a composition which is intended notably for the make-up of keratin fibres, particularly eyelashes or the hair, in forming drops at their tips upon its application, and which comprises at least one polymer having characteristics of viscoelasticity and of dynamic viscosity as defined above within the context of the method and as defined in the independent composition claims, said composition not containing any product having a viscoelasticity-modifying effect, which can prevent the formation of said drops, at the concentration used and said polymer being dispersed in a volatile solvent.

According to another embodiment of the invention, the invention relates also to a composition which is intended notably for the make-up of keratin fibres, particularly eyelashes or the hair, in forming drops at their tips upon its application, and which essentially consists of, or which consists of, said polymer or mixture of polymers selected from the family of hydroxylated polydimethylsiloxanes or non-hydroxylated polydimethylsiloxanes, and of their mixtures, and which has characteristics of viscoelasticity and of dynamic viscosity as defined above for the above composition or within the context of the method of make-up and as defined in the independent composition claim, said polymer or mixture of polymers being dispersed in a volatile solvent.

The advantageous characteristics of each one of these compositions according to the invention is the subject of the composition sub-claims, which are incorporated in their entirety by reference, these various embodiments being also described within the context of the make-up method, and which naturally apply to this second aspect of the invention covering the compositions.

According to a particularly advantageous variant, the composition of the invention contains, as polymers, a linear dimethiconol having a viscosity of around 6,400 Pa·s at 25° C. in solution in a volatile solvent comprising hexamethyldisiloxane.

The Examples which follow are given purely as an illustration of the invention. In the Examples, all the percentages are given by weight, the temperature is ambient temperature or is expressed in degrees Celsius, the pressure is atmospheric pressure, unless indications to the contrary.

### PRESENTATION OF THE FIGURES

The Examples are completed by FIGS. 1 to 6 which represent, respectively:

FIG. 1: the parameters of viscoelasticity of a dimethiconol polymer which can be used for the implementation of the invention (marketed by Dow Corning under the designation SGM36®),

FIG. 2: the viscosity curve of the same polymer,

FIG. 3: a photograph of a drop obtained in the goniometric study of the composition containing the dimethiconol polymer, for a contact time of 15 seconds, on an eyelash directed upwards,

FIG. 4: a photograph of the same drop as in FIG. 3 obtained in the goniometric study, but for a contact time of 90 seconds,

FIG. 5: a photograph of the same drop as that of FIG. 3 or 4, but for a contact time of 300 seconds, and

FIG. 6: a drop formed on an eyelash saturated with said composition and directed downwards after a contact time of 45 minutes.

### EXAMPLES

### I. Rheological Characterisations of Polymers

The characterisations of the viscoelasticity and of the viscosity of the polymers, were made at 25° C. and under atmospheric pressure on the pure polymer with the aid of a Rheometric Dynamic Analyser RDAII rheometer which is marketed by the company Rheometrics, USA, under the following conditions which are recommended in the use notice from the constructor.

1. Viscoelasticity

The moduli G′ and G″ are followed as a function of the frequency applied by means of said Rheometric Dynamic Analyser RDAII rheometer.

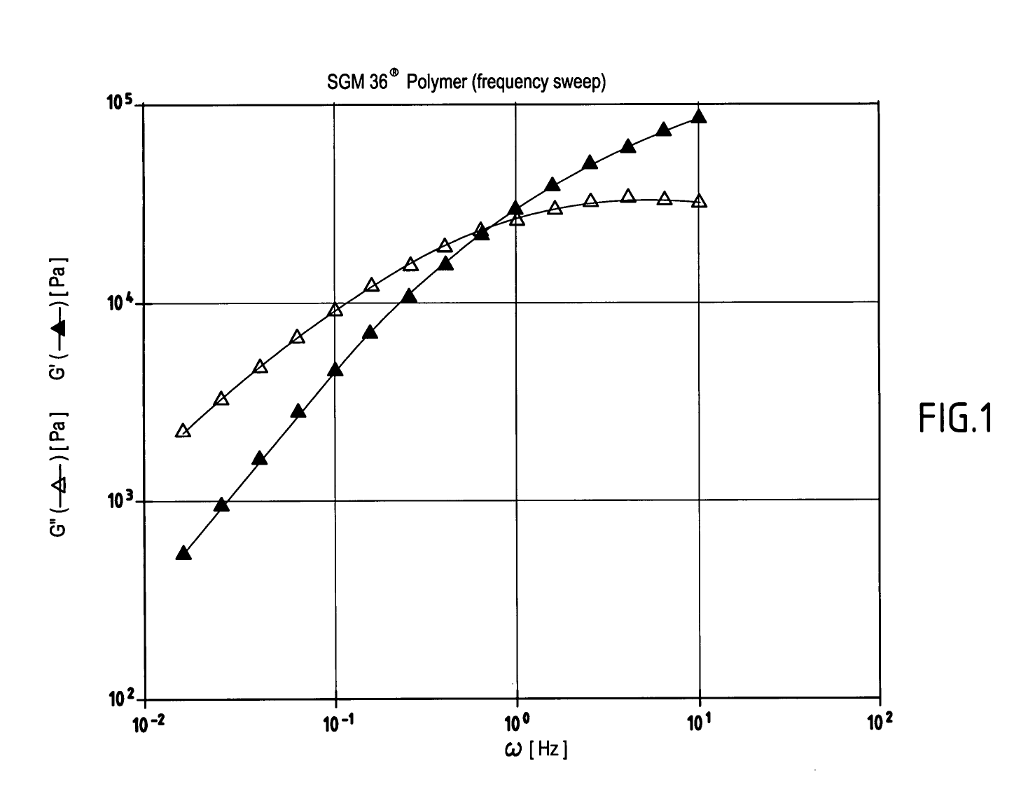

FIG. 1 attached is an example of curves which represent the evolution of G′ and G″ with the frequency ω for the dimethiconol SGM36® polymer from Dow Corning the point of intersection of the curves representing G′ and G″ of which correspond to a frequency of the order of 1 Hz.

2. Viscosity

The viscosity was studied with the aid of said rheometer.

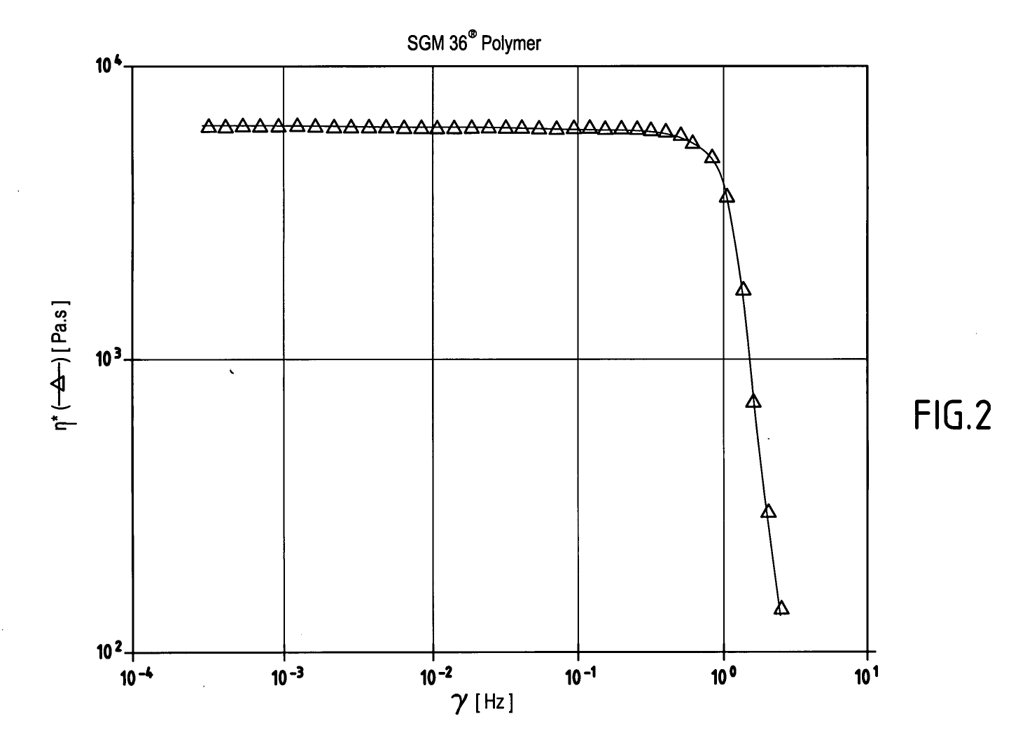

An example of a viscosity curve of the polymer suitable for the invention, namely the dimethiconol polymer referenced SGM36® from Dow Corning here is given in FIG. 2.

### II. Preparation and Evaluation de Compositions which can be Used According to the Invention

1. Preparation

The various compositions are all prepared with a Rayneri homogeniser.

19.2 parts by weight of dimethiconol SGM36® polymer are dissolved in 72.8 parts by weight of hexamethyldisiloxane solvent from Dow Corning, referenced Dow Corning 200 0.65 cts, with the aid of a homogeniser. 8 parts by weight of an anti-transfer additive is then added which reduces the sticking power, and which is here a mixture of cyclomethicone D5 and a dimethicone polymer cross-linked in vinyldimethicone, marketed under the trademark KSG15® mentioned above, and the agitation is maintained until a homogeneous and transparent viscous liquid is obtained.

The product thus obtained is ready for application.

2. Application

Two possible modes of application are distinguished in Example 2:

2.1. Application with a Threaded Stem

1. The product to be tested is contained in a 30 ml glass flask, reference 7409001263, from VG emballages.

2. False eyelashes of natural hair, trademark L.J.C., reference 63-200, are deposited on a Perspex column.

3. A threaded stem, reference 151 Silver Cap 500NS-140-3.250, from Henlopen, is obtained.

4. This stem (the threaded part) is immersed in the product to be tested, and the excess product is removed on the edge of the flask; the stem thus loaded with product in an amount which is sufficient to be deposited on the eyelashes, and just enough in order for it not to run from the stem before application; about 0.15 g is estimated to be the amount necessary of product to suitably load the threaded stem used.

5. The eyelashes are then brushed from the base towards the tip, once above, once below.

6. The stem is once again loaded as in [4] and the tips of the eyelashes are tapped in order to position the drops definitively. The optimum dimension of these drops is around 1 to 2 mm.

7. Drying takes place in a few minutes naturally in free air.

2.2 Application with a Brush

This variant, which consists in taking some product with the tip of a brush (lip brush type) and in depositing this product just at the tips of the eyelashes, enables the drops to be positioned precisely and their size to be modulated.

3. Evaluation

In addition to the visual appreciation of the formation of the drops, an evaluation by goniometry of the formation of the drops was also made under the following conditions:

The application is made with the aid of a standard mascara brush. The liquid is deposited on the dry eyelash or the eyelash saturated beforehand, at its free tip, with the solution to be tested.

The deposit is made either on the eyelash directed downwards, or on the eyelash directed upwards.

The acquisition of the images is made in static mode with the aid of a digital camera equipped with a 18-108/2.5 zoom functioning in macro mode. The focussing is made in the plane of the keratin fibre at a zoom/eyelash distance of about 160 mm with back lighting series of red-coloured diodes.

More specifically, this acquisition of images is firstly made in kinetic mode, so as to observe the evolution at short times (just after the deposit of the liquid on the eyelash), at the rate of one image every 15 seconds, and then in static mode with the view to observing the evolution of the form of the drops at long times with punctual taking of images.

The treatment of the images and the determination of the angle of contact are made with recourse to WinCalc® and WinGoutte® software and by means of a classical commercial goniometer which enables the angle of contact to be measured between the drop and the surface of the eyelash.

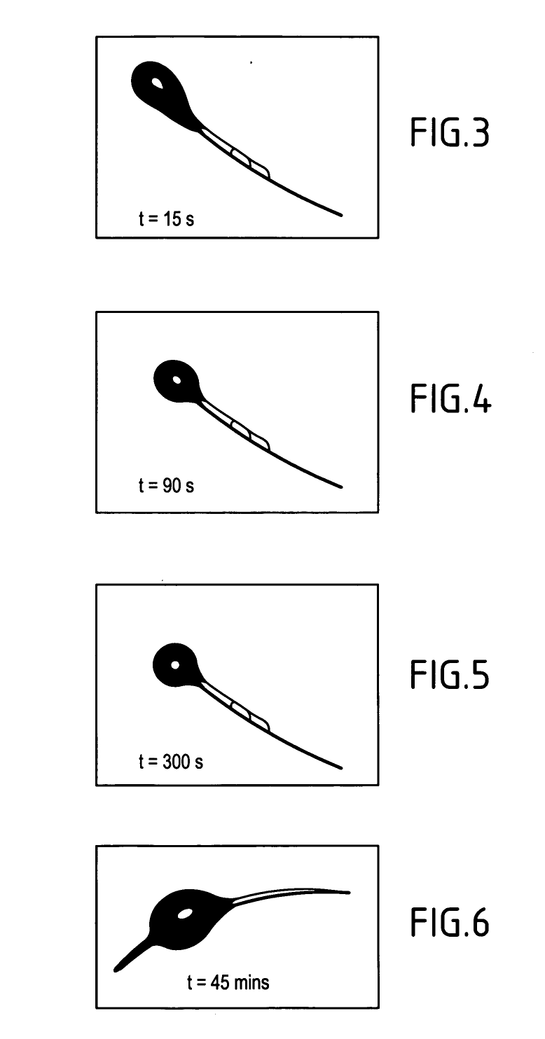

An example of the appearance of drops is given in FIG. 3, for a deposit at the tip of a dry eyelash directed upwards, after 15 seconds;

FIG. 4 represents the appearance of the drop similar to FIG. 3, but pour a residence time of 90 seconds, and this enables already observing the formation of the drop in a more circular way;

FIG. 5 represents the appearance of the drop after 300 seconds, and this enables observing the rapid formation of a spherical drop, with little evolution at long time, and therefore a very good stability; and

FIG. 6 represents another example of appearance of a drop on an eyelash directed upwards, saturated with the same composition, for a contact time of 45 minutes, where an excellent stability of the drop with time can still be observed.

Thus, the composition according to the invention enables relatively spherical drops to be formed which are stable with time and which are preferably transparent.

1-23. (canceled)

24. A method of make-up of keratin fibres intended to form drops on these fibres, comprising applying, onto said fibres, a composition containing 5 to 30% by weight of a polymer or mixture of polymers selected from the group consisting of dimethiconols and of their mixtures, and which has:
a viscoelasticity characterised by a conservation modulus G′ and a loss modulus G″, which are such that G′ be less than G″ for frequencies of lower than 0.3 Hz and greater than G″ for frequencies of higher than 3 Hz, the two curves representing G′ and G″ as a function of the frequency having a point of intersection in the interval between 0.3 and 3 Hz, and
a dynamic viscosity of between 4,000 and 10,000 Pa·s at 25° C.;
dispersed in a volatile solvent,
said composition not containing any product having a viscoelasticity-modifying effect, which can prevent the formation of said drops, at the concentration used.

25. The method according to claim 24, wherein said polymer is a linear dimethiconol having a viscosity of around 6,400 Pa·s at 25° C.

26. The method according to claim 24, wherein said volatile solvent is selected from a linear dimethicone having 2 to 9 silicon atoms, and a cyclomethicone having 3 to 8 silicon atoms.

27. The method according to claim 24, wherein said volatile solvent is hexamethyldisiloxane.

28. The method according to claim 24, wherein the concentration of polymer(s) is between 10 to 25% by weight with respect to the weight of the make-up composition.

29. The method according to claim 24, wherein the concentration of polymer is 15 to 25% by weight with respect to the weight of the make-up composition.

30. The method according to claim 24, wherein said composition further contains a product intended to reduce the sticky character of the drops.

31. The method according to claim 24, wherein said product is a mixture of a cyclomethicone D5 and a dimethicone polymer which is cross-linked by vinyldimethicone.

32. The method according to claim 24, wherein said keratin fibres are eyelashes.

33. The method according to claim 24, wherein said keratin fibres are the hair.

34. The method according to claim 24, wherein the composition contains a cosmetically-acceptable additive which is non-viscoelasticity-modifying at the concentration used.

35. A method of make-up of keratin fibres intended to form drops on these fibres, comprising applying, onto said fibres, a composition which essentially consists of, or which consists of, a dispersion in a volatile solvent of 5 to 30% by weight of a polymer or mixture of polymers selected from the group consisting of dimethiconols and of their mixtures, and which has:
a viscoelasticity characterised by a conservation modulus G′ and a loss modulus G″, which are such that G′ be less than G″ for frequencies of lower than 0.3 Hz and greater than G″ for frequencies of higher than 3 Hz, the two curves representing G′ and G″ as a function of the frequency having a point of intersection in the interval between 0.3 and 3 Hz, and
a dynamic viscosity of between 4,000 and 10,000 Pa·s at 25° C.

36. The method according to claim 35, wherein said polymer is a linear dimethiconol having a viscosity of around 6,400 Pa·s at 25° C.

37. The method according to claim 35, wherein said volatile solvent is selected from a linear dimethicone having 2 to 9 silicon atoms, and a cyclomethicone having 3 to 8 silicon atoms.

38. The method according to claim 35, wherein said volatile solvent is hexamethyldisiloxane.

39. The method according to claim 35, wherein the concentration of polymer(s) is between 10 to 25% by weight with respect to the weight of the make-up composition.

40. The method according to claim 35, wherein the concentration of polymer is 15 to 25% by weight with respect to the weight of the make-up composition.

41. The method according to claim 35, wherein said composition further contains a product intended to reduce the sticky character of the drops.

42. The method according to claim 35, wherein said product is a mixture of a cyclomethicone D5 and a dimethicone polymer which is cross-linked by vinyldimethicone.

43. The method according to claim 35, wherein said keratin fibres are eyelashes.

44. The method according to claim 35, wherein said keratin fibres are the hair.

45. The method according to claim 35, wherein the composition contains a cosmetically-acceptable additive which is non-viscoelasticity-modifying at the concentration used.

46. A composition which is intended notably for the make-up of keratin fibres, in forming drops at their tips upon its application, and which comprises 5 to 30% by weight of a polymer or mixture of polymers selected from the family of dimethiconols, and of their mixtures, and which has:
a viscoelasticity characterised by a conservation modulus G′ and a loss modulus G″, which are such that G′ be less than G″ for frequencies of lower than 0.3 Hz and greater than G″ for frequencies of higher than 3 Hz, the two curves representing G′ and G″ as a function of the frequency having a point of intersection in the interval between 0.3 and 3 Hz,
a dynamic viscosity of between 4,000 and 10,000 Pa·s at 25° C.,
said polymer or mixture of polymer being dispersed in a volatile solvent,
said composition not containing any product having a viscoelasticity-modifying effect, which can prevent the formation of said drops, at the concentration used.

47. The composition according to claim 46, wherein said polymer is a linear dimethiconol having a viscosity of around 6,400 Pa·s at 25° C.

48. The composition according to claim 46, wherein said volatile solvent is selected from a linear dimethicone having 2 to 9 silicon atoms, and a cyclomethicone having 3 to 8 silicon atoms.

49. The composition according to claim 46, wherein said volatile solvent is hexamethyldisiloxane.

50. The composition according to claim 46, wherein the concentration of polymer(s) is between 10 to 25% by weight with respect to the weight of the make-up composition.

51. The composition according to claim 46, wherein the concentration of polymer is 15 to 25% by weight with respect to the weight of the make-up composition.

52. The composition according to claim 46, wherein said composition further contains a product intended to reduce the sticky character of the drops.

53. The composition according to claim 52, wherein said product is a mixture of a cyclomethicone D5 and a dimethicone polymer which is cross-linked by vinyldimethicone.

54. The composition according to claim 46, wherein the composition contains a cosmetically-acceptable additive which is non-viscoelasticity-modifying at the concentration used.

55. The composition according to claim 46, wherein the polymer is a linear dimethiconol having a viscosity of around 6,400 Pa·s at 25° C. in solution in a volatile solvent comprising hexamethyldisiloxane.

56. A composition which is intended notably for the make-up of keratin fibres, in forming drops at their tips upon its application, and which essentially consists of, or which consists of, a dispersion in a volatile solvent of 5 to 30% by weight of a polymer or mixture of polymers selected from the family of dimethiconols, and of their mixtures, and which has:
a viscoelasticity characterised by a conservation modulus G′ and a loss modulus G″, which are such that G′ be less than G″ for frequencies of lower than 0.3 Hz and greater than G″ for frequencies of higher than 3 Hz, the two curves representing G′ and G″ as a function of the frequency having a point of intersection in the interval between 0.3 and 3 Hz,
a dynamic viscosity of between 4,000 and 10,000 Pa·s at 25° C.

57. The composition according to claim 56, wherein said polymer is a linear dimethiconol having a viscosity of around 6,400 Pa·s at 25° C.

58. The composition according to claim 56, wherein said volatile solvent is selected from a linear dimethicone having 2 to 9 silicon atoms, and a cyclomethicone having 3 to 8 silicon atoms.

59. The composition according to claim 56, wherein said volatile solvent is hexamethyldisiloxane.

60. The composition according to claim 56, wherein the concentration of polymer(s) is between 10 to 25% by weight with respect to the weight of the make-up composition.

61. The composition according to claim 56, wherein the concentration of polymer is 15 to 25% by weight with respect to the weight of the make-up composition.

62. The composition according to claim 56, wherein said composition further contains a product intended to reduce the sticky character of the drops.

63. The composition according to claim 62, wherein said product is a mixture of a cyclomethicone D5 and a dimethicone polymer which is cross-linked by vinyldimethicone.

64. The composition according to claim 56, wherein the composition contains a cosmetically-acceptable additive which is non-viscoelasticity-modifying at the concentration used.

65. The composition according to claim 56, wherein the polymer is a linear dimethiconol having a viscosity of around 6,400 Pa·s at 25° C. in solution in a volatile solvent comprising hexamethyldisiloxane.
